(19) United States
(12) Patent Application Publication
(10) Pub. No.: US 2019/0239769 A1
(43) Pub. Date: Aug. 8, 2019

(54) ELECTRONIC DEVICE INCLUDING DETACHABLE MEASUREMENT MODULE AND ATTACHMENT PAD

(71) Applicant: Samsung Electronics Co., Ltd., Gyeonggi-do (KR)

(72) Inventors: Minhyoung LEE, Gyeonggi-do (KR); Kyungho KIM, Gyeonggi-do (KR); Doyoon KIM, Gyeonggi-do (KR); Moorim KIM, Gyeonggi-do (KR); Sangjoon PARK, Gyeonggi-do (KR); Seongdae YANG, Gyeonggi-do (KR); Hayoul JUNG, Gyeonggi-do (KR); Woochul LEE, Gyeonggi-do (KR); Byunghun CHO, Gyeonggi-do (KR)

(21) Appl. No.: 16/270,736

(22) Filed: Feb. 8, 2019

(30) Foreign Application Priority Data
Feb. 8, 2018 (KR) 10-2018-0015635

(51) Int. Cl. A61B 5/0408; A61B 5/0432; A61B 5/00

(52) U.S. Cl. CPC A61B 5/04085; A61B 5/0432; H01F 7/02; A61B 5/0022; A61B 5/6833

(57) ABSTRACT

An electronic device and method are disclosed herein. The electronic device includes a housing, electrodes disposed on a face of the housing, and a processor which implements the method. The method includes in response to an electrocardiogram request, detecting a first signal using a first electrode and a fourth electrode from among the plurality of electrodes, detecting a second signal using a second electrode and the fourth electrode, detecting a third signal using a third electrode and the fourth electrode, and storing in the memory the first signal and the second signal as a first biological signal, the second signal and the third signal as a second biological signal and the third signal and the first signal as a third biological signal in association with the requested electrocardiogram measurement.

### CROSS-REFERENCE TO RELATED APPLICATION

This application is based on and claims priority under 35 U.S.C. § 119(a) of Korean Patent Application No. 10-2018-0015635, filed on Feb. 8, 2018, in the Korean Intellectual Property Office, the disclosure of which is incorporated herein by reference in its entirety.

### BACKGROUND

### 1. Field

Certain embodiments disclosed herein relate to an electronic device. For example, certain embodiments relate to a biological signal measurement device including a measurement module detachably provided on an attachment pad.

### 2. Description of the Related Art

Biological signals such as heart rate, heart rhythm, electrocardiogram (ECG), photoplethysmography (PPG), blood pressure, blood oxygen saturation, respiratory rate, blood glucose, and body heat may be used as various indexes for predicting information about the health status of a user (or a patient). Such information based on biological signals can be variously utilized not only for a patient's medical treatment purposes, but also for healthcare purposes. An electronic device attached to a user's body can be utilized to detect biological signals such as heart rate, heart rhythm, electrocardiogram (ECG), and photoplethysmogram (PPG).

### SUMMARY

In an embodiment, such a measurement device or electronic device may have a structure in which a circuit device and measurement electrodes (e.g., electrodes that are in contact with the user's body) are integrated in the form of a single module so as to be attached to the user's body. However, a measurement device in which a circuit device and measurement electrodes are integrated in the form of a single module may be limited in its ability to ensure a sufficient interval between the electrodes. For example, taking into consideration the bending of the user's body, as the measurement electrodes are disposed closer to each other, the measurement device can be more easily attached to the user's body. However, since measurement sites are closer to each other as the measurement electrodes are disposed closer to each other, the accuracy of the detected biological signal may be reduced. For example, a measurement device in which a circuit device and measurement electrodes are integrated into one module may be limited in ensuring measurement accuracy while miniaturizing the measurement device.

In another embodiment, the biological signal measurement device may have a structure in which a plurality of measurement electrodes attached to the user's body are disposed on the respective ones of a plurality of pads attached to the user's body and a circuit device is embedded in a snap structure that connects the plurality of pads. This structure may be advantageous in that it is easy to attach the structure to the user's body and in that it is possible to secure a certain interval between measurement electrodes. However, since the snap structure is disposed at a considerable height from the user's body, the measurement device may be uncomfortable for the user in the attached state.

In another embodiment, the biological signal measurement device may have a plurality of pads, each of which is provided with the measuring electrode(s), and each of the pads may be connected to the circuit device via conducting wire(s). In the measurement device having this structure, each pad can be easily attached to the user's body, and the number, the attachment positions, and the like of the measurement electrodes may be determined somewhat freely, which may allow the measurement device to have a high degree of accuracy in measurement. However, the structure of connecting a measurement electrode of the pad to the circuit device through conducting wire(s) is cumbersome to carry and is capable of being utilized in limited environments, such as a medical institution or a fitness center.

In some embodiments, the measurement device may include an attachment pad so that it can be attached to the user's body. Such an attachment pad should be generally discarded and replaced after being used once in consideration of hygiene and the like, but the measurement electrode or the like may be damaged in the process of replacing the attachment pad.

Certain embodiments are capable of providing an electronic device such as a biological signal measurement device, in which it is easy to replace an attachment pad and in which it is possible to prevent the measurement electrode(s) from being damaged in the replacement process.

Certain embodiments are capable of providing an electronic device such as a biological signal measurement device capable of improving the accuracy of biological signal measurement by ensuring sufficient space between the measurement electrodes while being miniaturized.

Certain embodiments are capable of providing an electronic device such as a biological signal measurement device, which is easy to carry so as to be capable of measuring a biological signal in various environments.

According to certain embodiments of the present disclosure, an electronic device is disclosed including a housing, a plurality of electrodes disposed on one face of the housing, at least one processor, and memory storing programming instructions executable by the at least one processor to cause the electronic device to: in response to receiving a request for an electrocardiogram measurement, detect a first signal using a first electrode and a fourth electrode from among the plurality of electrodes, detect a second signal using a second electrode and the fourth electrode, detect a third signal using a third electrode and the fourth electrode, and storing in the memory the first signal and the second signal as a first biological signal, the second signal and the third signal as a second biological signal and the third signal and the first signal as a third biological signal in association with the requested electrocardiogram measurement.

According to certain embodiments, an attachment pad for a biological signal measurement device is disclosed, the attachment pad including a pad body including a coupling portion for coupling to a module housing or a measurement module, and extension portions each extending away from the coupling portion, a coupling member provided in the coupling portion and disposed on a first face of the pad body, a plurality of terminals disposed in the coupling member and a plurality of measurement electrodes, each measurement electrode disposed on each of the extension portions respectively, each of the extension portions disposed on a second face of the pad body oriented away from the first face of the pad body, wherein each of the measurement electrodes is electrically connected to one of the plurality of terminals. According to certain embodiments, an electronic device or a biological signal measurement device is configured to detachably attach a measurement module and an attachment pad using magnetic force. Thus, the attachment pad can be easily replaced while stably maintaining the coupled state. According to certain embodiments, the attachment pad can be fabricated or deformed into various shapes while securing a sufficient interval between the measurement electrodes. Thus, the attachment pad can be easily attached to the user's body. For example, it is possible to increase the accuracy of biological signal measurement by securing a sufficient interval between the measurement electrodes while miniaturizing the measurement module. According to certain embodiments, since the measurement module is miniaturized, carrying the measurement module is convenient. Further, it is possible to measure a biological signal by selecting an attachment pad having an appropriate shape according to the user's need.

### BRIEF DESCRIPTION OF THE DRAWINGS

The above and other aspects, features, and advantages of the present disclosure will be more apparent from the following detailed description taken in conjunction with the accompanying drawings, in which:

FIG. 1 is a block diagram illustrating an electronic device in a network environment, including a detachable measurement module and an attachment pad, according to certain embodiments;

FIG. 2 is a block diagram illustrating a biological signal measurement device according to certain embodiments;

FIG. 3 is an exploded perspective view illustrating a biological signal measurement device according to certain embodiments;

FIG. 4 is a perspective view illustrating a biological signal measurement device according to certain embodiments in the assembled state;

FIG. 5 is an exploded perspective view illustrating a measurement module of a biological signal measurement device according to certain embodiments;

FIG. 6 is an exploded perspective view illustrating a first electrode structure in a measurement module of a biological signal measurement device according to certain embodiments;

FIG. 7 is a bottom view illustrating a measurement module of a biological signal measurement device according to certain embodiments;

FIG. 8 is a side view illustrating the measurement module of the biological signal measurement device according to certain embodiments;

FIG. 9 is an exploded perspective view illustrating a coupling member in an attachment pad of a biological signal measurement device according to certain embodiments;

FIG. 10 is a plan view illustrating the coupling member in the attachment pad of the biological signal measurement device according to certain embodiments;

FIG. 11 is a bottom view illustrating the coupling member in the attachment pad of the biological signal measurement device according to certain embodiments;

FIG. 12 is an exploded perspective view illustrating a pad body in an attachment pad of a biological signal measurement device according to certain embodiments;

FIG. 13 is a plan view illustrating the pad body in the attachment pad of the biological signal measurement device according to certain embodiments;

FIG. 14 is a view illustrating various shapes of an attachment pad of a biological signal measurement device according to certain embodiments;

FIG. 15 is a flowchart for describing a biological signal measurement method of an electronic device according to certain embodiments; and

FIG. 16 is a view illustrating biological signals measured or determined through an electronic device according to certain embodiments.

### DETAILED DESCRIPTION

As the present disclosure allows for various changes and numerous embodiments, some example embodiments will be described in detail with reference to the accompanying drawings. However, it should be understood that the present disclosure is not limited to the specific embodiments, but the present disclosure includes all modifications, equivalents, and alternatives within the present disclosure.

Although ordinal terms such as “first” and “second” may be used to describe various elements, these elements are not limited by the terms. The terms are used merely for the purpose to distinguish an element from the other elements. For example, a first element could be termed a second element, and similarly, a second element could be also termed a first element without departing from the present disclosure. As used herein, the term “and/or” includes any and all combinations of one or more associated items.

Further, the relative terms “a front surface”, “a rear surface”, “a top surface”, “a bottom surface”, and the like which are described with respect to the orientation in the drawings may be replaced by ordinal numbers such as first and second. In the ordinal numbers such as first and second, their order are determined in the mentioned order or arbitrarily and may not be arbitrarily changed if necessary.

In the present disclosure, the terms are used to describe specific embodiments, and are not intended to limit the present disclosure. As used herein, the singular forms are intended to include the plural forms as well, unless the context clearly indicates otherwise. In the description, it should be understood that the terms “include” or “have” indicate existence of a feature, a number, a step, an operation, a structural element, parts, or a combination thereof, and do not previously exclude the existences or probability of addition of one or more another features, numeral, steps, operations, structural elements, parts, or combinations thereof.

Unless defined differently, all terms used herein, which include technical terminologies or scientific terminologies, have the same meaning as that understood by a person skilled in the art to which the present disclosure belongs. Such terms as those defined in a generally used dictionary are to be interpreted to have the meanings equal to the contextual meanings in the relevant field of art, and are not to be interpreted to have ideal or excessively formal meanings unless clearly defined in the present specification.

In the present disclosure, an electronic device may be a random device, and the electronic device may be called a terminal, a portable terminal, a mobile terminal, a communication terminal, a portable communication terminal, a portable mobile terminal, a touch screen or the like.

For example, the electronic device may be a smartphone, a portable phone, a game player, a TV, a display unit, a heads-up display unit for a vehicle, a notebook computer, a laptop computer, a tablet Personal Computer (PC), a Personal Media Player (PMP), a Personal Digital Assistants (PDA), and the like. The electronic device may be implemented as a portable communication terminal which has a wireless communication function and a pocket size. Further, the electronic device may be a flexible device or a flexible display device.

The electronic device may communicate with an external electronic device, such as a server or the like, or perform an operation through an interworking with the external electronic device. For example, the electronic device may transmit an image photographed by a camera and/or position information detected by a sensor unit to the server through a network. The network may be a mobile or cellular communication network, a Local Area Network (LAN), a Wireless Local Area Network (WLAN), a Wide Area Network (WAN), an Internet, a Small Area Network (SAN) or the like, but is not limited thereto.

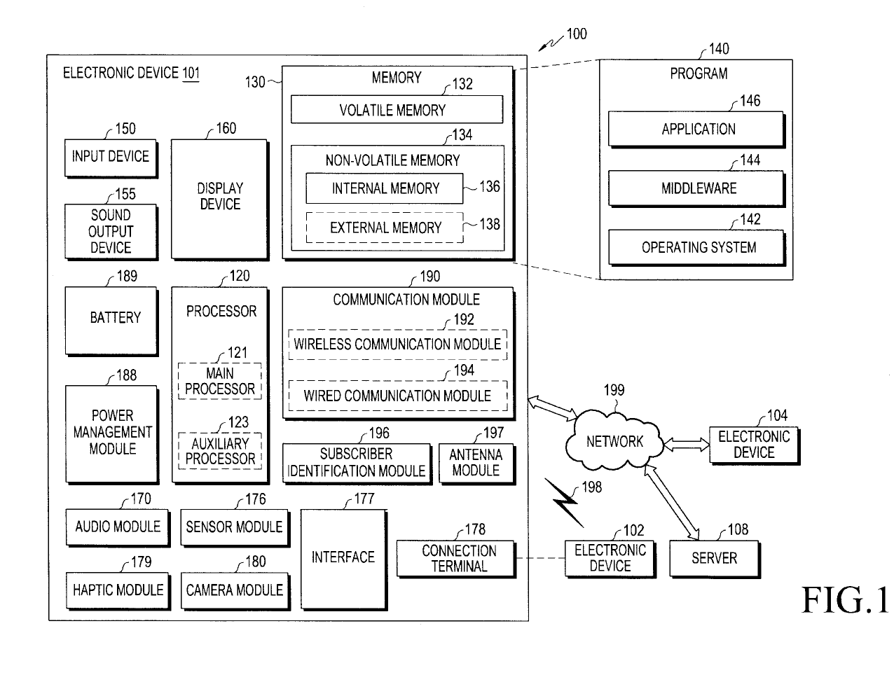

FIG. 1 is a block diagram illustrating an electronic device 101 in a network environment 100 according to certain embodiments. Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input device 150, a sound output device 155, a display device 160, an audio module 170, a sensor module 176, an interface 177, a haptic module 179, a camera module 180, a power management module 188, a battery 189, a communication module 190, a subscriber identification module (SIM) 196, or an antenna module 197. In some embodiments, at least one (e.g., the display device 160 or the camera module 180) of the components may be omitted from the electronic device 101, or one or more other components may be added in the electronic device 101. In some embodiments, some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may load a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134. The non-volatile memory may include internal memory 136 and external memory 138.

The program 140 may be stored in the memory 130 as software, and may include, for example, an operating system (OS) 142, middleware 144, or an application 146.

The input device 150 may receive a command or data to be used by other component (e.g., the processor 120) of the electronic device 101, from the outside (e.g., a user) of the electronic device 101. The input device 150 may include, for example, a microphone, a mouse, a keyboard, or a digital pen (e.g., a stylus pen).

The sound output device 155 may output sound signals to the outside of the electronic device 101. The sound output device 155 may include, for example, a speaker or a receiver. The speaker may be used for general purposes, such as playing multimedia or playing record, and the receiver may be used for an incoming calls. According to an embodiment, the receiver may be implemented as separate from, or as part of the speaker.

The display device 160 may visually provide information to the outside (e.g., a user) of the electronic device 101. The display device 160 may include, for example, a display, a hologram device, or a projector and control circuitry to control a corresponding one of the display, hologram device, and projector. According to an embodiment, the display device 160 may include touch circuitry adapted to detect a touch, or sensor circuitry (e.g., a pressure sensor) adapted to measure the intensity of force incurred by the touch.

The audio module 170 may convert a sound into an electrical signal and vice versa. According to an embodiment, the audio module 170 may obtain the sound via the input device 150, or output the sound via the sound output device 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The antenna module 197 may transmit or receive a signal or power to or from the outside (e.g., the external electronic device) of the electronic device 101. According to an embodiment, the antenna module 197 may include an antenna including a radiating element implemented by a conductive material or a conductive pattern formed in or on a substrate (e.g., PCB). According to an embodiment, the antenna module 197 may include a plurality of antennas. In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

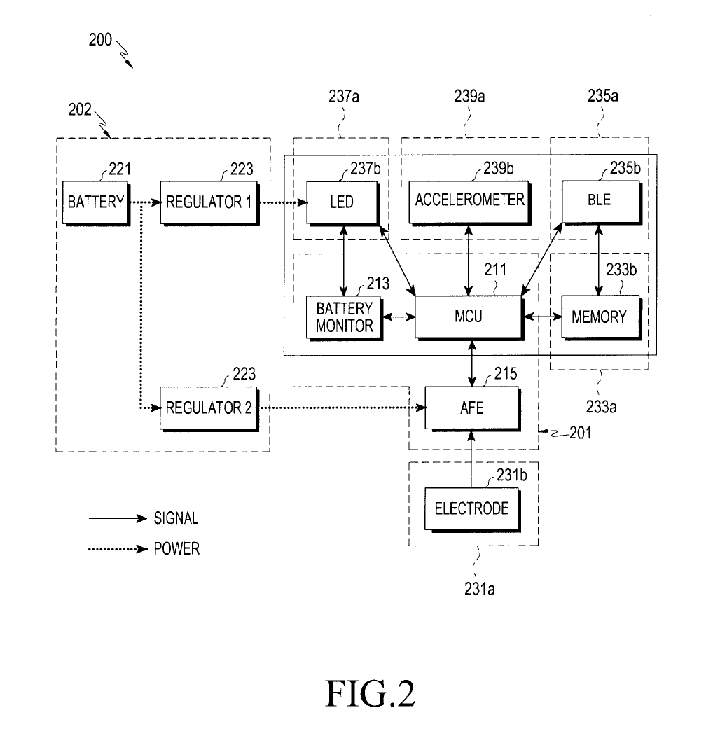

FIG. 2 is a block diagram illustrating a biological signal measurement device 200 according to certain embodiments.

The biological signal measurement device 200 may include some or all of the components including, for example, the electronic device 101 of FIG. 1. Referring to FIG. 2, the biological signal measurement device 200 may include a control unit 201, a power supply unit 202, and a mounting unit 231a, and may further include a storage unit 233a, a communication unit 235a, a display unit 237a, and a measurement unit 239a in some embodiments.

According to certain embodiments, the control unit 201 may include a Main Control Unit (MCU) 211, a battery monitor 213, and an Analog Front End (AFE) 215. The control unit 211 may include, for example, the processor 120 of FIG. 1, and may perform the control of the entire biological signal measurement device 200. In an embodiment, the battery monitor 213 may measure the remaining capacity of a battery 221 included in the power supply unit 202, or the like. In another embodiment, the AFE 215 may digitize a biological signal such as an analog voltage signal detected through the mounting unit 231a, and may transmit the digitized biological signal to the control unit 211.

According to certain embodiments, the power supply unit 202 may include a battery 221 and at least one regulator 223 (e.g., regulator 1 and regulator 2), and in some embodiments, may include the power management module 188 and the battery 189 of FIG. 1. In an embodiment, the battery 221 may supply power for driving the biological signal measurement device 200, and may include a non-rechargeable primary battery, a rechargeable secondary battery, or a fuel cell. In another embodiment, the regulator 223 may convert the power of the battery 221 into a voltage suitable for driving the biological signal measurement device 200 (e.g., the control unit 211), and may supply the voltage.

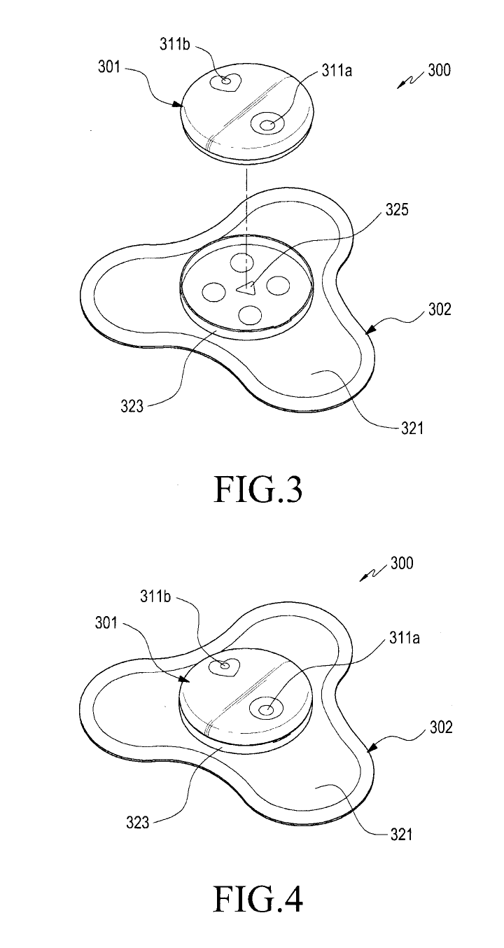

According to certain embodiments, the control unit 211 and the power supply unit 202 may be embedded in substantially one housing (e.g., the module housing 301 illustrated in FIG. 3, which will be described later). In some embodiments, the housing, in which the power supply unit 202 and the like are embedded, may include a switch device (e.g., an operation unit 311a in FIG. 3) for turning power on/off or initiating/terminating measurement. The switch device may be a part of the power supply unit 202 or the control unit 211.

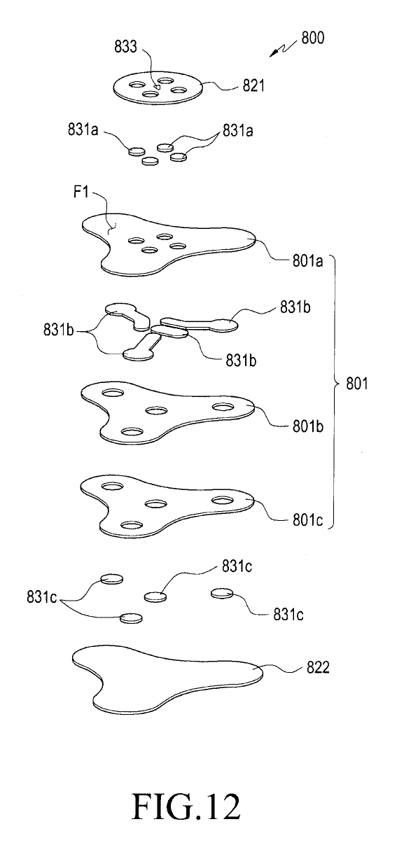

According to certain embodiments, the mounting unit 231a may provide an implement for attaching the biological signal measurement device 200 to the body of a user or a patient, and may be in direct contact with the user's body so as to transmit a current or voltage signal to the control unit 211 (e.g., the AFE 215). For example, the mounting unit 231a may include an electrode(s) 231b in contact with the user's body, and the electrode 231b (e.g., a measurement electrode or a third wiring electrode 831c in FIG. 12) may be electrically connected to the AFE 215.

According to certain embodiments, the control unit 211 may generate information on the electrocardiogram, the heartbeat, and the like of the user to whom the biological signal measurement device 200 is attached on the basis of the digital signal received through the AFE 215. In some embodiments, information (e.g., first measurement information) generated by the control unit 211 may be stored in the storage unit 233a. For example, the storage unit 233a may store information generated by the control unit 211 by including a memory 233b (e.g., the memory 130 in FIG. 1).

According to certain embodiments, the information generated by the control unit 211 or the information stored in the storage unit 233a may be transmitted to another electronic device (e.g., the electronic device 102 in FIG. 1) via the communication unit 235a. In another embodiment, the information generated by the control unit 211 or the information stored in the storage unit 233a may be transmitted to yet another electronic device (e.g., the electronic device 104 in FIG. 1) or a server (e.g., the server 108 in FIG. 1) via the communication unit 235a and via a network (e.g., the network 199 in FIG. 1). For example, the communication unit 235a is capable of transmitting generated information or stored information to another electronic device directly or via a network by including Bluetooth Low Energy (BLE) 235b. In another embodiment, when the communication unit 235a maintains a state of being connected with another electronic device, either directly or via a network, the control unit 211 may transmit the generated information to the other electronic device without storing it in the storage unit 233a.

According to certain embodiments, the display unit 237a may output information on the state of the biological signal measurement device 200 under the control of the control unit 211, for example. According to an embodiment, the display unit 237a is capable of visually displaying the remaining charge of the battery, the state of attachment to the user's body (e.g., whether or not a biological signal is detectable), whether or not communication with another electronic device or the like is possible, and the like by including a light source (e.g., a Light-Emitting Diode (LED) 237b). For example, the LED 237b may provide various kinds of information to the user through the color, the blinking period, and the like of the output light. Although not illustrated, the display unit 237a may output various kinds of information through a speaker (e.g., the sound output device 155 in FIG. 1), a vibration device (e.g., the haptic module 179 in FIG. 1), a display (e.g., the display device 160 in FIG. 1), and the like, in addition to the above-mentioned light source. The configuration of the display unit 237a, as described above, may be appropriately selected in consideration of the size and usage of the biological signal measurement device 200, the attachment position of the biological signal measurement device 200 on the user's body, and the like.

According to certain embodiments, the measurement unit 239a is capable of measuring a motion (e.g., an amount of motion) of a user who wears or attaches an electronic device (e.g., the biological signal measurement device 200). For example, the measurement unit 239a may include at least an accelerometer 239b, and in some embodiments, the measurement unit 239a may include a gyro sensor, an atmospheric pressure sensor, a temperature sensor, or a humidity sensor (e.g., the sensor module 176 in FIG. 1) so as to detect the user's momentum, the environment at the time of measuring a biological signal, and the like. The control unit 211 may generate second measurement information on the user's momentum, temperature, humidity, etc., detected by the measurement unit 239a, and may store the second measurement information in the storage unit 233a. The first measurement information and the second measurement information stored in the biological signal measurement device 200 (e.g., the storage unit 233a) are used as basic data capable of analyzing a health condition such as the user's physical strength.

FIG. 3 is an expanded perspective view illustrating a biological signal measurement device 300 according to certain embodiments. FIG. 4 is a perspective view illustrating the biological signal measurement device 300 according to certain embodiments in the assembled state.

Referring to FIGS. 3 and 4, an electronic device such as the biological signal measurement device 300 (e.g., the biological signal measurement device 200 in FIG. 2) may include a detachable housing (e.g., a module housing 301) and an attachment pad 302. According to an embodiment, the attachment pad 302 may provide an implement for attaching the biological signal measurement device 300 to the user's body. In some embodiments, the attachment pad 302 may be limited with respect to a number of times it can be attached to the user's body, due to weakening of attachment force over successive attachments, as a result of loss of adhesion, and/or hygienic problems such as contamination and infection. Accordingly, a medical institution may, in principle, prescribe only a single use of the attachment pad 302 thereof. The module housing 301 may include circuits and/or devices for performing biological (e.g., biometric) signal measurement, such as a control unit 201 and a power supply unit 202 in FIG. 2, and may be coupled to the attachment pad 302 by magnetic force. For example, when the attachment pad 302 is to be replaced due to loss of adhesion over successive reattachments, the measurement module (e.g., as part of the module housing 301) may be re-used by being coupled to a new attachment pad.

According to certain embodiments, the bottom face of the module housing 301 (e.g., the face facing the attachment pad 302) may be formed generally flat, and the upper face may be formed in a domed shape. For example, the module housing 301 is capable of accommodating the above-described control unit, power supply unit, and the like therein by forming a dome-shaped internal space. According to an embodiment, the module housing 301 may include an operation unit 311a configured to operate a switch device (or the like) of a power supply unit (e.g., the power supply unit 202 in FIG. 2), and an output unit 311b configured to output light, an image, sound, or the like, as provided via a display unit (e.g., the display unit 237a in FIG. 2) to the outside. Since the operation unit 311a and the output unit 311b are disposed on the upper face of the module housing 301, the module housing 301 may be exposed to the outside even when the module housing 301 is coupled to the attachment pad 302.

According to certain embodiments, the attachment pad 302 may include a pad body 321 made of a flexible sheet or the like, and a coupling member 323 provided on one face of the pad body 321. The coupling member 323 may enclose at least a part of the module housing 301, such as, for example, the bottom face of the module housing 301. For example, the coupling member 323 may include a substantially circular fence shape protruding from the one face of the pad body 321, so that the module housing 301 can provide a degree of fixing force while guiding coupling.

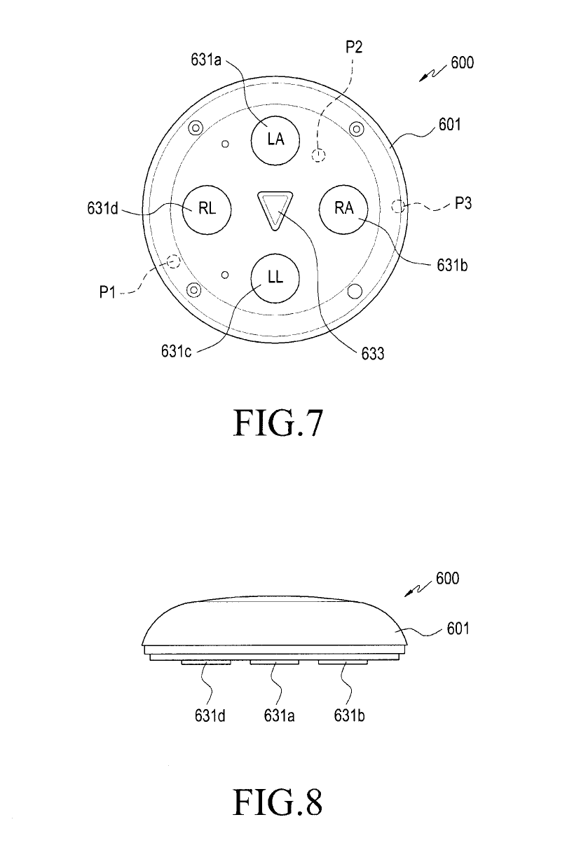

According to certain embodiments, the biological signal measurement device 300 may include an alignment key structure to set a direction in which the module housing 301 couples to the attachment pad 302. For example when the module housing 301 is aligned in a predetermined direction relative to the attachment pad 302, the module housing may be stably coupled to the attachment pad 302 (e.g., the coupling member 323). In some embodiments, the alignment key structure may be configured to include a combination of a first alignment key (e.g., a first alignment key 633 of FIG. 7 to be described below) protruding from the bottom face of the module housing 301, and a second alignment key (e.g., an alignment recess denoted by reference numeral “325”) in a depressed shape in the coupling member 323. The alignment key structure may be designed in various shapes and positions, and may guide the module housing 301 in an intended direction in order to couple with the attachment pad 302.

According to certain embodiments, an adhesive may be applied to the other face of the pad body 321 (e.g., the face opposite the face on which the coupling member 323 is disposed). For example, the other face of the pad body 321 (e.g., the bottom face of the pad body 321, which is not visible in FIG. 3) may therefore be attachable to the user's body. In order to attach to the user's body, the pad body 321 may be formed of a flexible sheet or the like, and may have various shapes which may help conformity to curvature, movements and/or bending of the user's body. For example, the pad body 321 may be formed as to be easily attachable to the user's body, based on the material and shape in which it is formed. In some embodiments, the region of the pad body 321 to which the module housing 301 is coupled may have a certain degree of rigidity (such as at the coupling member 323). For example, the pad body 321 may stably maintain the coupling state with the module housing 301 while being flexibly deformed in order to substantially correspond to the bending of the body.

According to certain embodiments, the module housing 301, or at least the bottom face of the module housing 301, may have a regular polygonal or circular shape in plan view. The shape of the module housing is capable of providing an environment in which a larger number of electrodes (e.g., electrodes for biological signal detection or electric signal transmission) is capable of being disposed in a limited area (e.g., the area of the bottom face of the module housing 301). In biological signal detection, as the number of electrodes is increased, the accuracy of measurement can be increased. For example, when at least a pair of electrodes among the plurality of electrodes is in contact with the user's body, it is possible to detect biological signals through the corresponding electrodes. In some embodiments, when a plurality of respective electrodes are in contact with the user's body, two arbitrarily selected electrodes may be set as leads. For example, when three electrodes are used for biological signal measurement, three pairs of electrode combinations (e.g., leads) are possible, and the accuracy of the detected information is capable of being diversified or improved by detecting biological signals through each electrode combination.

According to certain embodiments, the electrodes disposed in the module housing 301 are capable of providing a path for transmitting a voltage or current signal or the like substantially corresponding to a detected biological signal, and a measurement electrode(s) that come in contact with the user's body may be provided on the attachment pad (e.g., on the other face of the pad body 321). For example, the measurement electrodes may be electrically connected to the module housing 301 through wires provided inside the pad body 321 or the coupling member 323. Since the pad body 321 is capable of being flexibly deformed corresponding to the bending of the body, it is possible to provide an environment in which a sufficient interval can be secured between the measurement electrodes. The arrangement of the measurement electrodes, the electrical connection structure to the module housing 301, and the like will be described in more detail with reference to FIG. 12 or the like.

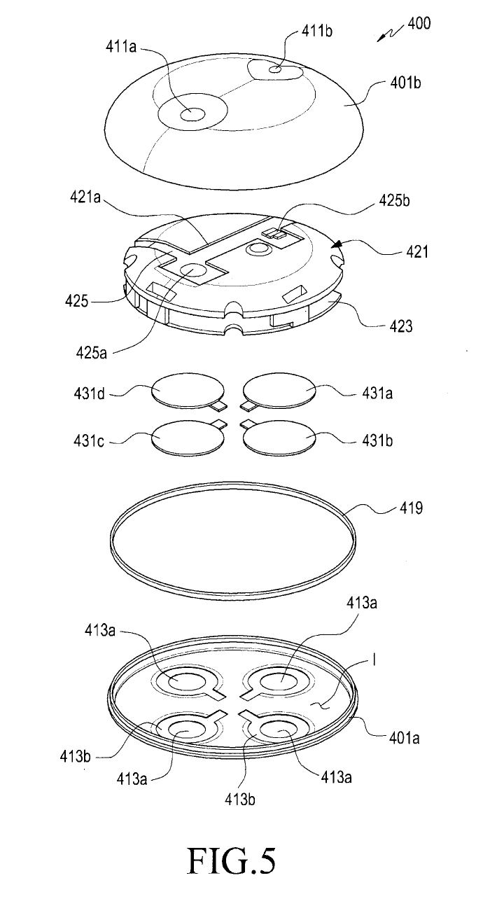

FIG. 5 is an expanded perspective view illustrating a measurement module 400 of a biological signal measurement device according to certain embodiments of the present disclosure.

Referring to FIG. 5, the measurement module 400 of the biological signal measurement device (e.g., the biological signal measurement device 300 in FIG. 3), according to certain embodiments, may accommodate various circuit devices or the like in an interior hollow or space (e.g., the inner space of the module housing 301 in FIG. 3) formed by a combination or coupling of a first case member 401a and a second case member 401b, and may include a first electrode 431a, a second electrode 431b, a third electrode 431c, or a fourth electrode 431d exposed to the outer face of the first case member 401a (e.g., the bottom face of the module housing 301 of FIG. 3). In some embodiments, the fourth electrode 431d may be provided as a reference electrode for measuring a biological signal (e.g., electrocardiogram).

According to certain embodiments, the outer face of the first case member 401a may form the bottom face of the measurement module 400, and the first case member 401a may include a plurality of first openings 413a in order to expose the first to fourth electrodes 431a to 431d disposed therein. A stepped face 413b may be formed around the first openings 413a, and may be disposed on the inner face I of the first case member 401a. Further, the edges of the first to fourth electrodes 431a to 431d may be fixed to the stepped faces 413b. For example, the first to fourth electrodes 431a to 431d may be mounted on or fixed to the inner face I of the first case member 401a, and may be exposed to the outside of the measurement module 400 through the first openings 413a. In some embodiments, the first to fourth electrodes 431a to 431d may be positioned at least in part to be substantially coplanar with the outer face of the first case member 401a. In other embodiments, the first to fourth electrodes 431a to 431d may actually protrude from the outer face of the first case member 401a by a predetermined height.

According to certain embodiments, the second case member 401b may include an operation unit 411a (e.g., the operation unit 311a in FIG. 3) configured to operate a switch device, or the like, and an output unit 411b (e.g., the output unit 311b in FIG. 3) configured to output light or sound. According to an embodiment, the second case member 401b may provide an interior hollow or space that accommodates various circuitry and/or devices (e.g., the control unit 201 and the power supply unit 202 in FIG. 2). For example, the second case member 401b may have a substantially polyhedral or domed shape, and the inner space may be closed when the first case member 401a is coupled to the second case member 401b.

According to certain embodiments, the measurement module 400 may include a support member 421, and a circuit board 423 disposed inside the second case member 401b. In an embodiment, the circuit board 423 is fixed on or above the inner face of the second case member 401b via the support member 421. The circuit device (s) of the measurement module 400 may be mounted or disposed on the circuit board 423. According to an embodiment, the support member 421 may include a support structure on which the circuit board 423 is supported or fixed, and although not illustrated in the drawings, a battery (e.g., the battery 189 or 221 in FIG. 1 or FIG. 2) may be disposed between the circuit board 423 and the support member 421. For example, the circuit board 423 may be coupled to the support member 421 and may be disposed so as to partially surround the space in which the battery is mounted.

According to certain embodiments, the biological signal measurement device (e.g., the biological signal measurement device 300 in FIG. 3), for example, the measurement module 400, may include a flexible printed circuit board 425 extending from the circuit board 423. A switch member 425a or a light-emitting element 425b may be mounted on the flexible printed circuit board 425, and may be electrically connected to the circuit board 423 (e.g., the control unit 201 in FIG. 2). According to an embodiment, the flexible printed circuit board 425 may be mounted on the other face of the support member 421 (e.g., the face facing the second case member 401b in FIG. 5) and may be disposed such that the switch member 425a corresponds to the operation unit 411a or such that the light-emitting element 425b corresponds to the output unit 411b. For example, when viewed with reference to the support member 421, the flexible printed circuit board 425 may be disposed so as to be directed away from the bottom face of the measurement module 400 (e.g., the outer face of the first case member 401a) and to face the inner face of the second case member 401b. According to an embodiment, on the other face of the support member 421 (e.g., the face facing the first case member 401a), a wiring recess 421a having a depth corresponding to the thickness of the flexible printed circuit board 425 (or deeper than the thickness of the flexible printed circuit board 425) may be provided. For example, in the state of being mounted on or fixed to the support member 421, the flexible printed circuit board 425 is capable of being protected from interference from other structures by being located in the wiring recess 421a.

According to certain embodiments, the switch member 425a may include a dome switch, a tact switch, or a touch sensor, and may be disposed to correspond to the operation unit 411a. For example, when the user operates the operation unit 411a, the switch member 425a may generate an on/off signal of the measurement module 400. According to the settings of the control unit or the memory (e.g., the control unit 201 or the storage unit 233a in FIG. 2) of the measurement module 400, the switch member 425a may generate a signal for changing the operation mode of the measurement module 400 or changing the output method of the display unit. In another embodiment, when the measurement module 400 includes a communication module (e.g., the communication unit 235a in FIG. 2), the measurement module 400 may transmit data relating to measured or stored biological information, or may reset the operation mode or the communication mode according to the presets of a processor or the operation of the switch member 425a.

According to certain embodiments, the light-emitting element or device 425b is an example of an output device that substantially forms the display unit 237a in FIG. 2, and may visually output the status information of the measurement module 400 or the results of biological signal detection by a combination of a color of light, a blinking signal, and the like. In some embodiments, the light-emitting element 425b may be replaced by a display or a sound output device, or may be installed together with a display or a sound output device. For example, the measurement module 400 may output operation state information or information on the results of biological signal detection or the like, not only through the color of light or a blinking signal, but also in the form of an image, a character, sound or the like.

According to certain embodiments, the second case member 401b may be coupled to face the first case member 401a in the state in which the support member 421 or the circuit board 423 is accommodated therein. For example, the space in which the circuit board 423 is accommodated may be substantially sealed by the first case member 401a and the second case member 401b. According to an embodiment, when coupling the first case member 401a and the second case member 401b, a fastening member such as a screw is fastened from the first case member 401a to sequentially penetrate the circuit board 423 and the support member 421 so as to be bound to the inner face of the second case member 401b. According to another embodiment, in the state in which the first case member 401a and the second case member 401b are coupled to each other, the first to fourth electrodes 431a to 431d may be positioned to face at least a part of the circuit board 423. Although not illustrated, the first to fourth electrodes 431a to 431d may be electrically connected to a circuit device (e.g., the AFE 215 in FIG. 2) provided on the circuit board 423 via elastic bodies such as pogo pins and C-clips.

According to certain embodiments, the measurement module 400 is capable of blocking the introduction of foreign matter, moisture, or the like into the inner space (e.g., the inner space of the module housing 301 in FIG. 3) by including a first waterproofing member 419. For example, the first waterproofing member 419 may have a shape corresponding to the edge of the first case member 401a (e.g., an O-ring), and may be interposed between the first case member 401a and the second case member 401b. When the first case member 401a and the second case member 401b are bound together by the fastening member or the like, the first waterproofing member 419 may form a sealing structure or a waterproofing structure by being pressed to a certain degree between the first case member 401a and the second case member 401b.

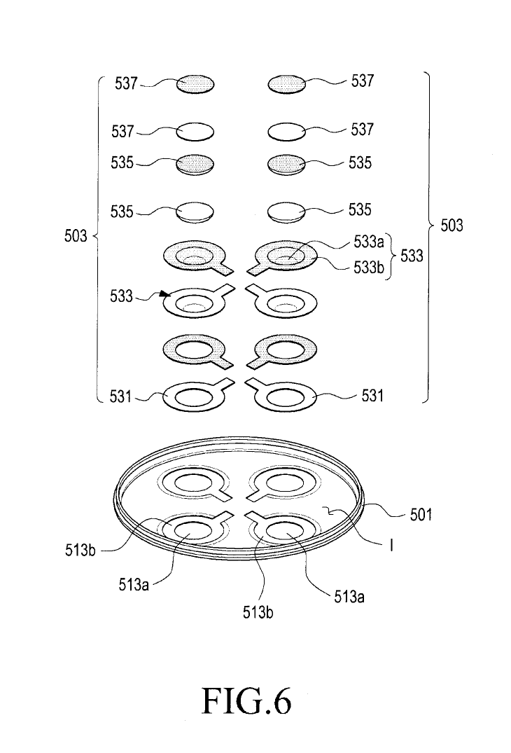

According to an embodiment, the measurement module 400 may include a permanent magnet (e.g., a permanent magnet 535 in FIG. 6) so as to be coupled to an attachment pad (e.g., the attachment pad 302 in FIG. 3). The arrangement structure, such as a permanent magnet or the like, will be described with reference to FIG. 6.

FIG. 6 is an expanded perspective view illustrating the arrangement of electrodes 503 in a measurement module of a biological signal measurement device according to certain embodiments.

Referring to FIG. 6, the measurement module (e.g., the measurement module 400 in FIG. 6) of the biological signal measurement device described above may include a permanent magnet(s) 535 disposed in at least one of the electrodes 503 (e.g., the first to fourth electrodes 431a to 431d in FIG. 5). In some embodiments, the permanent magnets 535 may be disposed on the respective ones of the electrodes 503 (e.g., the first to fourth electrodes 431a to 431d in FIG. 5). For example, each of the electrodes 503 may include a first electrode plate 533 formed using an electrically conductive material and a permanent magnet 535 disposed on or in the first electrode plate 533. In an embodiment, the first electrode plate 533 may include an accommodation recess 533a formed in the inner face thereof, and a flange 533b disposed circumferentially around the accommodation recess 533a. For example, the permanent magnet 535 may be received in the accommodation recess 533a, on the inner face of the first electrode plate 533.

According to certain embodiments, the first electrode plate 533 may be formed of a magnetic substance (e.g., stainless steel), and the permanent magnet 535 may be affixed or otherwise secured in the accommodation recess 533a, even if no separate fixing or attachment implemented is provided. For example, the permanent magnet 535 may be attached or fixed to the first electrode plate 533 or the accommodation recess 533a by magnetic force. In another embodiment, the electrode 503 may more stably fix the permanent magnet 535 in the accommodation recess 533a by further including a second electrode plate 537 coupled to the inner face of the first electrode plate 533. The second electrode plate 537 may be made of a magnetic substance as to be coupled to the first electrode plate 533 through the permanent magnet 535. In some embodiments, the second electrode plate 537 may be directly coupled to the inner face of the first electrode plate 533 as to close the accommodation recess 533a and fix the permanent magnet 535.

According to certain embodiments, the electrode(s) 503 may be mounted on the inner face I of the case member 501 (e.g., the first case member 401a in FIG. 5). The case member 501 may include a plurality of first openings 513a, and stepped faces 513b, each of which may be formed around a respective openings 513a, and on the inner face I. The stepped faces 513b may be formed as to substantially correspond to the flanges 533b. For example, the electrode(s) 503 may be fixed to the inner face I of the case member 501 by coupling the flange(s) 533b to the stepped face(s) 513b. When the electrodes 503, for example, the first electrode plates 533, are mounted on or fixed to the stepped faces 413b, the outer faces of the first electrode plates 533 (which correspond to the accommodation recesses 533a) may be exposed to the outer face of the case member 501 through the first openings 513a. The region exposed through each first opening 513a (e.g., a part of the outer face of each first electrode plate 533) may be substantially coplanar with the outer face of the case member 501, or may partially protrude from the outer face of the case member 501.

According to certain embodiments, a biological signal measurement device (e.g., the biological signal measurement device 300 in FIG. 4 or the measurement module 400 in FIG. 5) may include first adhesive members 531 that attach the flanges 533b of the module housing (e.g., the module housing 301), for example, the case member 501. The first adhesive members 531 may include, for example, a piece of double-sided tape, and may attach the electrodes 503 (e.g., the first electrode plates 533) to the first openings 513a so as to seal the first openings 513a, serving therefore as waterproofing structures. In an embodiment, the first adhesive members 531 may be formed in a shape corresponding to the flanges 533b or the stepped faces 513b, and may substantially attach the flanges 533b to the stepped faces 513a.

According to certain embodiments, since the permanent magnets 535 are disposed in the electrodes 503, the structure of the measurement module (e.g., the module housing 301 in FIG. 3 or the measurement module 400 in FIG. 5) may be advantageously simplified and/or miniaturized. For example, since a separate structure to be coupled with an attachment pad (e.g., the attachment pad 302 of FIG. 3) is substantially unnecessary (e.g., since binding force is provided using the permanent magnet 535), it is possible to increase the utilization efficiency of the space inside the measurement module. As the utilization efficiency of the space inside the measurement module increases, further miniaturization of at least the measurement module is made possible, and thus, it is possible to dispose a larger-capacity battery in a measurement module of the same size.

In a specific embodiment of the present disclosure, a structure using magnetic force (e.g., the permanent magnet 535) as an implement for coupling the measurement module to the attachment pad is disclosed, but the present disclosure is not limited thereto. The measurement module may be combined with the attachment pad through, for example, a snap-fit structure using a combination of a hook (or an elastic body) and a recess, a structure in which a lock-releasing button is combined with the snap-fit structure, and a rotational coupling structure (e.g., screw-coupling). As described above, the coupling structure between a measurement module and an attachment pad (e.g., the module housing 301 and the attachment pad 302 in FIG. 3) may be appropriately selected in consideration of the size (e.g., the utilization efficiency of the inner space), shape, or structural stability of a biological signal measurement device (e.g., the biological signal measurement device 300 in FIG. 3), the alignment direction of a measurement module, and the like.

FIG. 7 is a bottom view illustrating a measurement module 600 of a biological signal measurement device according to certain embodiments of the present disclosure. FIG. 8 is a side view illustrating the measurement module 600 of the biological signal measurement device according to certain embodiments of the present disclosure.

Referring to FIGS. 7 and 8, the measurement module 600 (e.g., the module housing 301 in FIG. 3) of the biological signal measurement device, according to certain embodiments of the present disclosure, may include first to fourth electrodes 631a to 631d (e.g., the first to fourth electrodes 431a to 431d in FIG. 5) which may be exposed to or towards a first face of the housing 601 (e.g., the outer face of the first case member illustrated in FIG. 4 (e.g., the face directed away from the inner face I in the first case member 401a in FIG. 4), and a first alignment key 633 disposed on the first face of the housing 601.

According to certain embodiments, a polygon may be formed by combining straight lines drawn to connect two adjacent electrodes among the first to fourth electrodes 631a to 631d. For example, each of the first to fourth electrodes 631a to 631d illustrated in FIG. 7 may be arranged so as to form vertices substantially disposed in the shape of a square. In another embodiment, the first face of the measurement module 600 may be substantially disposed in the shape of a circle, and the first to fourth electrodes 631a to 631d may be arranged at equal angular intervals in the circumferential direction of the first face of the measurement module 600.

As mentioned above, the number and arrangement of the first to fourth electrodes 631a to 631d may vary. However, considering that the measurement module 600 has a rigid structure and is attached to the user's body, the area of the measurement module 600 (e.g., the area of the face on which the first to fourth electrodes 631a to 631d are disposed) may be limited. Therefore, the number of electrodes, and the arrangement of the first to fourth electrodes 631a to 631d may be appropriately selected in consideration of the area of the portion of the measurement module 600 (or the biological signal measurement device including the measurement module 600) that can be stably attached to the user's body.

According to certain embodiments, four electrodes (e.g., the first to fourth electrodes 631a to 631d) are disposed, and a pair of arbitrarily selected electrodes among the first to fourth electrodes 631a to 631d may be combined to detect a biological signal. For example, the first to fourth electrodes 631a to 631d are defined as an RL (Right Leg) electrode (e.g., the fourth electrode 631d), an LA (Left Arm) electrode (e.g., the first electrode 631a), an RA (Right Arm) electrode (e.g., the second electrode 631b), and an LL (Left Leg) electrode (e.g., the third angle 631c), the RL electrode may be utilized as a reference electrode, and each of an LL-RA electrode pair, an RA-LA electrode pair, and an LA-LL electrode pair may form a lead that detects a biological signal. In some embodiments, at least one of the electrode pairs listed above may detect a biological signal.

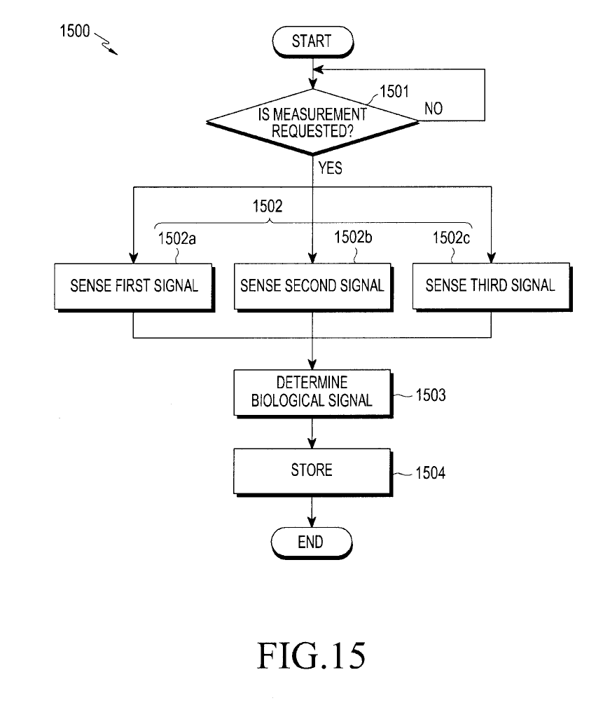

According to certain embodiments, an electronic device (e.g., a processor (e.g., processor 120 of FIG. 1) of the measurement module 600) may identify an input or request associated with an electrocardiogram measurement of a living body, may sense a signal using the first to fourth electrodes 631a to 631d on the basis of the input or request, and may determine the sensed signal as a biological signal associated with the electrocardiogram. The processor of the electronic device may store at least a part or one of the determined biological signal(s) in a memory (e.g., the memory 130 of FIG. 1) as at least a piece of measurement information of an electrocardiogram measurement. In some embodiments, at least a piece of the measurement information on an electrocardiogram measurement may be transmitted to another electronic device (e.g., the electronic device 102 or 104 in FIG. 1) or stored in a server (the server 108 in FIG. 1) through, for example, a communication module (e.g., the communication module 190 of FIG. 1 or the communication unit 235a in FIG. 2). An operation of detecting or measuring a biological signal using the first to fourth electrodes 631a to 631d or an electrode pair implemented by a combination of the first to fourth electrodes 631a to 631d will be described in more detail with reference to FIG. 15.

According to certain embodiments, some of the electrode pairs listed above may detect a biological signal, and the remaining electrode pairs may output a current signal or the like that stimulates the body. The “current signal that stimulates the body” may be provided for a treatment purpose. In another embodiment, when the “current signal that stimulates the body” may interfere with biological signal detection, current signals for biological signal detection and body stimulation may be alternatively or periodically alternately output.

In the embodiment, although it is described that “the first to fourth electrodes of the measurement module detect a biological signal”, it is noted that the first to fourth electrodes 631a to 631d are substantially a part of a path for transmitting a voltage or current signal corresponding to a detected biological signal. For example, a measurement electrode(s) (e.g., a third wiring electrode 831c in FIG. 12) of an attachment pad, which will be described later, actually comes into contact with the user's body to detect a biological signal, and the measurement electrode may be electrically connected to one of the first to fourth electrodes 631a-631d. In another embodiment, the “measurement electrodes” may be interpreted to mean including the first to fourth electrodes or the third wiring electrode 831c of FIG. 12, or to mean including a wiring path (e.g., the second wiring electrode 831b in FIG. 12) connecting the first to fourth electrodes and the third wiring electrode 831c. In the following description, “the electrodes that detect a biological signal” will be described again. However, as described above, an electrode in direct contact with the user's body and an electrode not in contact with the user's body may be easily distinguished through the entire description of embodiments, reference drawings, respective embodiments, and the like.

According to certain embodiments, the first alignment key 633 may establish a direction for coupling the measurement module 600 to an attachment pad (e.g., the attachment pad 302 in FIG. 3). The first alignment key 633 may have a polygonal shape (e.g., that of an isosceles triangle) that protrudes from a first face (e.g., the bottom face) of the measurement module 600 and is directional. The first alignment key 633 may be engaged with a second alignment key (e.g., the alignment recess 325 in FIG. 3) formed on a coupling member. For example, the second alignment key formed on the coupling member may have a shape corresponding to the first alignment key 633, and the measurement module 600 may be coupled with the coupling member in the direction in which the first alignment key 633 and the second alignment key of the coupling member are engaged with each other.

According to certain embodiments, the first alignment key 633 and the corresponding second alignment key may be provided in various shapes and positions. For example, the first alignment key 633 of the measurement module 600 may be formed in a recess shape, and the second alignment key formed on the coupling member may be formed in a protrusion shape. In another embodiment, the first alignment key 633 or the second alignment key may have a right-triangular shape. In another embodiment, when the first face of the measurement module 600 is a regular polygonal or circular shape, the first alignment key 633 may be positioned at any location other than the center (e.g., a position indicated by “P1”, “P2”, or “P3” on the first face of the measurement module 600). In another embodiment, when each of the first to fourth electrodes 631a to 631d is connected to any of the third wiring electrodes 831c of FIG. 12, the first alignment key 633 may have a regular polygonal shape corresponding to the number of the electrodes 631a to 631d. For example, when four first to fourth electrodes 631a to 631d are disposed in the measurement module 600 and an electrode (e.g., the fourth electrode 631d) is connected to any of the third wiring electrodes among the third wiring electrodes 831c, the first alignment key 633 may have a square shape.

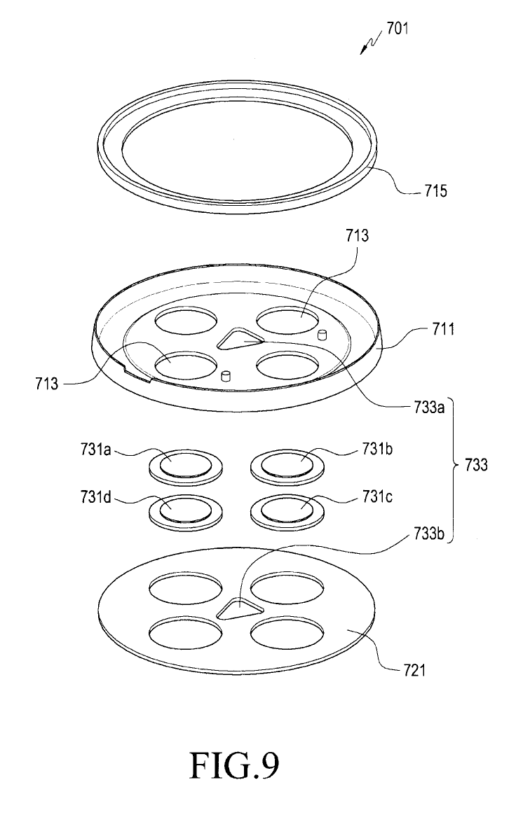

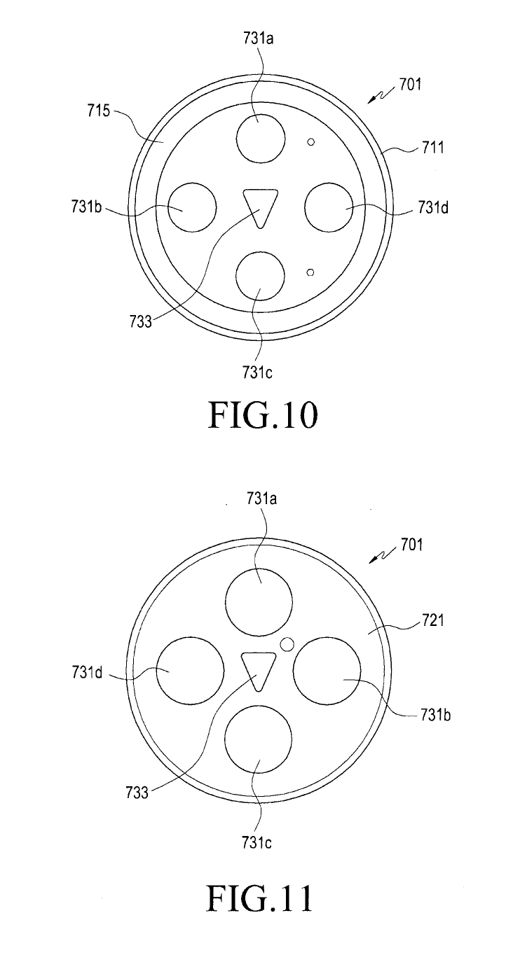

FIG. 9 is an expanded perspective view illustrating a coupling member 701 in an attachment pad of a biological signal measurement device according to certain embodiments. FIG. 10 is a plan view illustrating the coupling member 701 in the attachment pad of the biological signal measurement device according to certain embodiments. FIG. 11 is a bottom view illustrating the coupling member 701 in the attachment pad of the biological signal measurement device according to certain embodiments.

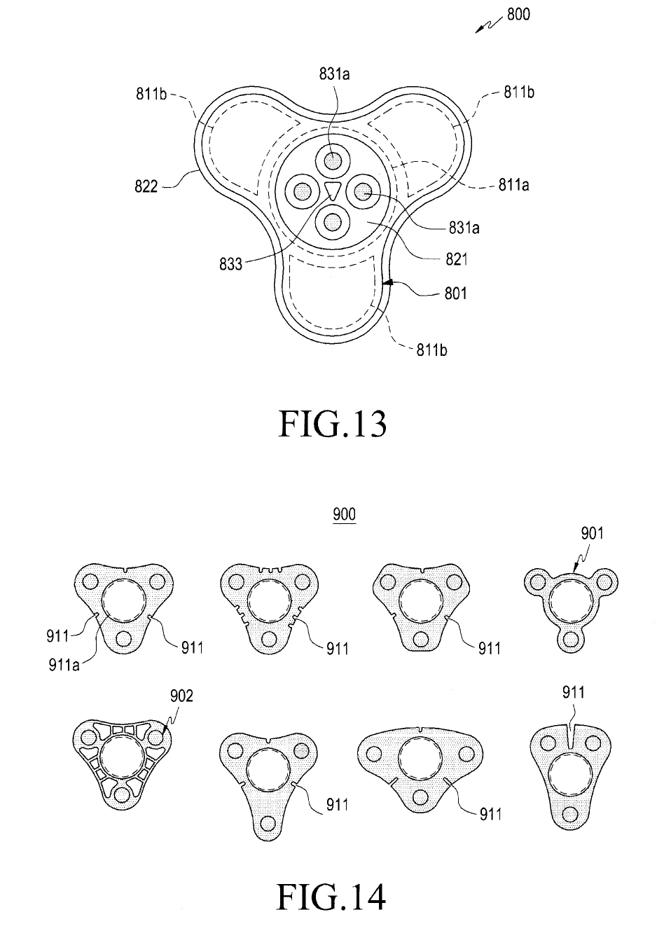

As described with reference to FIG. 3, the coupling member (e.g., the coupling member 323 of FIG. 3) of the biological signal measurement device, according to certain embodiments, may be formed as a part of the attachment pad (e.g., the attachment pad 302 in FIG. 3), and may be mounted on the first face (e.g., a first face F1 in FIG. 13) of the pad body (e.g., the pad body 321 in FIG. 3). Referring to FIG. 9, the coupling member 701 (e.g., the coupling member 323 in FIG. 3) may be formed in a shape that encloses a portion of the measurement module (e.g., the module housing 301 in FIG. 3 or the measurement module 400 in FIG. 5). In an embodiment, the coupling member 701 may include a seating plate 711 and a second waterproofing member 715.

According to certain embodiments, the coupling member 701 may further include first to fourth terminals 731a to 731d, which correspond to the respective ones of the first to fourth electrodes (e.g., the first to fourth electrodes 431a to 431d in FIG. 5) of the measurement module, and a second adhesive member 721 that attaches the seating plate 711 to the pad body. As will be described later, the first to fourth terminals 731a to 731d may be electrically connected to a first wiring electrode (e.g., the first wiring electrode 831a in FIG. 12) disposed substantially in the pad body, or may be a part of the first wiring electrode. The second adhesive member 721 may include a piece of double-sided tape disposed or attached to a first face (e.g., the first face F1 in FIG. 13) of the pad body.

According to certain embodiments, the seating plate 711 may be formed in a shape corresponding to at least a portion (e.g., the bottom face) of the measurement module (e.g., the measurement module 400 in FIG. 5), and may surround at least a part of the side face of the measurement module by including a fence structure formed at the edge thereof. For example, the seating plate 711 may be formed in a shape that encloses or receives a portion of the measurement module. According to an embodiment, the second waterproofing member 715 may be formed generally in the shape of a closed curve corresponding to the edge of the seating plate 711, and may be mounted inside a space in which the measurement module is accommodated (e.g., a space or hollow formed by a fence structure). For example, when the measurement module is accommodated in the seating plate 711 (or when the measurement module is coupled with the seating plate), the second waterproofing member 715 is capable of blocking intrusion of foreign matter, moisture, or the like into the space between the seating plate 711 and the measurement module (e.g., between the module housing 301 and the coupling member 323 in FIG. 3).

According to certain embodiments, second openings 713 may be formed through the seating plate 711 in the space in which the measurement module is accommodated. The second openings 713 may be formed substantially at positions corresponding to the first to fourth electrodes (e.g., the first to fourth electrodes 431a to 431d in FIG. 5) of the measurement module. In another embodiment, a first alignment hole 733a may be formed through the seating plate 711 in the space in which the measurement module is accommodated. The first alignment hole 733a may include at least a portion of a second alignment key 733 (e.g., the alignment recess 325 in FIG. 3) corresponding to the first alignment key of the measurement module (e.g., the first alignment key 633 in FIG. 7).

According to certain embodiments, the first to fourth terminals 731a-731d may be disposed in one of the second openings 713, respectively. For example, a plurality of the first to fourth terminals 731a to 731d may be mounted on the bottom face of the seating plate 711 to be exposed to the space in which the measurement module is accommodated through the second openings 713. In an embodiment, the first to fourth terminals 731a to 731d may be mounted on the seating plate 711 via other adhesive members to close the second openings 713. For example, the first to fourth terminals 731a to 731d may be mounted on the seating plate 711 via other adhesive members to form waterproofing structures on the second openings 713. In another embodiment, when the measurement module (e.g., the measurement module 400 in FIG. 5) is coupled to the coupling member 701, each of the first to fourth terminals 731a to 731d may be in electrical contact with one of the electrodes of the measurement module (e.g., the first to fourth electrodes 431a to 431d in FIG. 5).

According to certain embodiments, the first to fourth terminals 731a to 731d may include a conductive material or a magnetic substance. As described above, each of the first to fourth terminals 731a to 731d may be made of a conductive material, and may be in electrical contact with one of the electrodes of the measurement module (e.g., the first to fourth electrodes 431a to 431d in FIG. 5). According to an embodiment, the first to fourth terminals 731a to 731d may be made of a magnetic material, and may generate an attractive force with the first to fourth electrodes of the measurement module using the magnetic force of a permanent magnet (e.g., the permanent magnet 535 in FIG. 6). For example, the first to fourth terminals 731a-731d may couple and fix the measurement module (e.g., the module housing 301 in FIG. 3 or the measurement module 400 in FIG. 5) to the seating plate 711 using magnetic force while providing an electrical connection.

According to certain embodiments, the second adhesive member 721 may be formed of a piece of double-sided tape or the adhesive applied to the bottom face of the seating plate 711. The second adhesive member 721 may attach the seating plate 711 to a pad body (e.g., the pad body 321 in FIG. 3). In an embodiment, the second adhesive member 721 may include a second alignment hole 733b aligned with the first alignment hole 733a. For example, the first alignment hole 733a and the second alignment hole 733b may be combined to form a second alignment key (e.g., the alignment recess 325 in FIG. 3) corresponding to a first alignment key (e.g., the first alignment key 633 in FIG. 7). In another embodiment, the second adhesive member 721 may be provided on a first face of the pad body (e.g., the first face F1 in FIG. 13) rather than on the seating plate 711, and in some embodiments, the second adhesive member 721 may be provided on each of the seating plate 711 and the pad body. A pad body in a biological signal measurement device according to certain embodiments of the present disclosure will be described with reference to FIG. 12 and the like.

FIG. 12 is an expanded perspective view illustrating a pad body 801 in an attachment pad of a biological signal measurement device according to certain embodiments. FIG. 13 is a plan view illustrating the pad body 801 in the attachment pad of the biological signal measurement device according to certain embodiments.

Referring to FIGS. 12 and 13, the pad body 801 of the attachment pad 800 (e.g., the pad body 321 in FIG. 3) may be made of a sheet or the like that can be flexibly deformed to correspond to the contours and/or bending of the human body, and is capable of coupling the attachment pad 800 (e.g., attachment pad 302 in FIG. 3) with the coupling member 701 illustrated in FIG. 9 and the like. In an embodiment, the attachment pad 800 may include an elastic material. It is noted that FIGS. 12 and 13 illustrate the attachment pad 800 in the state in which the coupling member is omitted. The pad body 801 may include a base sheet 801a, a plurality of adhesive layers 801b and 801c, a measurement electrode(s), a wiring structure, and the like.

According to certain embodiments, the base sheet 801a is a flexible sheet that substantially forms the outer shape of the pad body 801, and conceals the measurement electrode(s) or the wiring structure so as to prevent the electrodes from being exposed to an external environment. In some embodiments, a second adhesive member 821 (e.g., the second adhesive member 721 in FIG. 9) may be provided on the upper face of the base sheet 801a (e.g., the first surface F1 of the pad body). The second adhesive member 821 may be provided on one or each of the coupling member 701 and the pad body 801 of FIG. 9. In some embodiments, when the second adhesive member 821 includes a second alignment hole 833 (e.g., the second alignment hole 733b in FIG. 9), the second alignment hole 833 may have a recessed shape, closed in the direction in which it is attached to the base sheet 801a. According to an embodiment, the base sheet 801a may be formed of a single-sided sticker. For example, an adhesive for fixing the wiring structure or the like may be applied to the bottom face of the base sheet 801a.

According to certain embodiments, the plurality of adhesive layers may include a first adhesive layer 801b and a second adhesive layer 801c. In an embodiment, the first adhesive layer 801b may include a piece of double-sided tape directly attached to the bottom face of the base sheet 801a. The above-mentioned wiring structure (e.g., a second wiring electrode 831b to be described later) and the like may be at least partially fixed between the base sheet 801a and the first adhesive layer 801b. In another embodiment, the second adhesive layer 801c may include a pressure-sensitive adhesive applied to the first adhesive layer 801b, and may directly attach the pad body 801 or a biological signal measurement device (e.g., the biological signal measurement device 300) to the user's body. For example, the second adhesive layer 801c may be an adhesive layer so as to be in direct contact with the user's body.

According to certain embodiments, the pad body 801 may further include a low-adhesion protective film 822. The low-adhesion protective film 822 is a film attached to the second adhesive layer 801c and is capable of preventing the second adhesive layer 801c from becoming polluted during the course of manufacturing, circulating, or storing the pad body 801 or the attachment pad 800. For example, when the attachment pad 800 is actually used, the low-adhesion protective film 822 may be removed from the pad body.

According to certain embodiments, in a plan view, the pad body 801 may include a coupling portion 811a and extension portions 811b. According to an embodiment, the coupling portion 811a means the region in which a coupling member (e.g., the coupling member 701 in FIG. 9) is disposed, and the second adhesive member 821 may be disposed on the coupling portion 811a. The extension portions 811b may extend from the coupling portion 811a in different directions, respectively. Each of the extension portions 811b may be provided as a region in which one of the measurement electrodes (e.g., the third wiring electrodes 831c to be described later) is disposed. For example, the extension portions 811b are capable of improving accuracy or the like in biological signal detection by securing an interval between the measurement electrodes.

According to certain embodiments, the wiring structure may include first wiring electrodes 831a and second wiring electrodes 831b, and is capable of electrically connecting the measurement electrode(s) (e.g., a third wiring electrode 831c to be described later) to a measurement module (e.g., the measurement module 400 in FIG. 5). According to an embodiment, the first wiring electrodes 831a may be disposed in the through holes formed in the base sheet 801a or the second adhesive member 821, and may be in electrical contact with or may be attached to terminals (e.g., the first to fourth terminals 731a to 731d in FIG. 9). For example, the second wiring electrodes 831b may have a double-sided tape structure (e.g., adhesiveness) and may be conductive. For example, the first wiring electrodes 831a may be disposed in the coupling portion 811a. As described above, each of the first wiring electrodes 831a may be a portion of one of the above-mentioned first to fourth terminals (e.g., the first to fourth terminals 731a to 731d in FIG. 9), or each of the first to fourth wiring terminals may be provided on a portion of one of the first wiring electrodes 831a. According to another embodiment, the second wiring electrodes 831b may be made of silver or silver chloride and may have conductivity and a certain degree of flexibility. The second wiring electrodes 831b may extend from the respective ones of the first wiring electrodes 831a. In some embodiments, one end of some of the second wiring electrodes 831b may be positioned on one of the extension portions 811b, and the other end of each of the second wiring electrodes 831b may be positioned on the coupling portion 811a. According to an embodiment, at least a part of the wiring structure, for example, the third wiring electrodes 831c, may be disposed between the base sheet 801a and the first adhesive layer 801b.

According to certain embodiments, when the measurement module (e.g., the measurement module 400 in FIG. 5) includes four first to fourth electrodes 431a to 431d, three extension portions 811b may be provided. The number of first wiring electrodes 831a or third wiring electrodes 831c may correspond to the number of first to fourth electrodes of the measurement module (e.g., the first to fourth electrodes 431a to 431d in FIG. 5). According to an embodiment, some of the second wiring electrodes 831b may extend from any one of the first wiring electrodes 831a in the coupling portion 811a, and the end of each of the second wiring electrodes 831b may be positioned on one of the extension portions 811b. The third wiring electrodes 831c, each having an end positioned on one of the extension portions 811b, may be connected to the LL (Left Leg) electrode, the RA (Right Arm) electrode, and the LA (Left Arm) electrode, which form a measurement lead, among the first to fourth electrodes of the measurement module (e.g., the first to fourth electrodes 631a to 631d in FIG. 7). For example, the second wiring electrodes 831b (e.g., the measurement electrodes), each having an end positioned on one of the extension portions 811b, is capable of transmitting a substantially detected biological signal or a voltage or current signal corresponding to the detected biological signal. According to another embodiment, any one of the second wiring electrodes 831b may extend from a remaining one of the first wiring electrodes 831a, and an end of the second wiring electrode 831b may be positioned in the coupling portion 811a. For example, any one of the third wiring electrodes 831c may be positioned in the coupling portion 811a, and may be connected to the reference electrode (e.g., the fourth electrode 631d or the RL electrode in FIG. 7) among the first to fourth electrodes of the measurement module. In an embodiment, the end of the second wiring electrode 831b connected to the reference electrode or the third wiring electrode 831c connected to the reference electrode may be positioned in the center of the coupling portion 811a.

According to certain embodiments, the measurement electrode(s) provided in the pad body 801 may include the third wiring electrodes 831c, which are respectively provided at the ends of the second wiring electrodes 831b. The third wiring electrodes 831c may be exposed to the outside on a second face directed away from the first face F1 of the pad body 801 (e.g., on the second adhesive layer 801c). For example, the third wiring electrodes 831c may be exposed to the outside of the attachment pad 800 or the pad body 801 in a direction different from that of the first terminal 731a, the second terminal 731b, the third terminal 731c, or the fourth terminal 731d (e.g., the opposite direction) in FIG. 10.

According to certain embodiments, when the second adhesive layer 801c is attached to the user's body (skin), the third wiring electrode(s) 831c may be in direct contact with the user's body. The third wiring electrode(s) 831c may be made of a conductive hydrogel and may stably maintain contact with the user's body. In an embodiment, a biological signal may be detected by the third wiring electrode(s) 831c and may be transmitted to a measurement module (e.g., the first to fourth electrodes 431a to 431d in FIG. 5) via the second wiring electrode(s) 831b and the first wiring electrode(s) 831a (or the first to fourth terminals 731a to 731d in FIG. 9).

According to certain embodiments, since the third wiring electrodes 831c are disposed on the respective ones of the extension portions 811b, the third wiring electrodes 831c may be arranged with a interval therebetween that is larger than the first wiring electrodes 831a located in the coupling portion 811a or the first to fourth electrodes 631a to 631d of FIG. 7. For example, the attachment pad 800 is capable of securing sufficient space between the measurement electrodes (e.g., the third wiring electrodes 831c) so as to create an environment capable of stably detecting a biological signal. In an embodiment, any one of the third wiring electrodes 831c may be located in the center of the coupling portion 811a. For example, any one of the third wiring electrodes 831c may be disposed on an end of the second wiring electrode located in the coupling portion 811a among the second wiring electrodes 831b and may be electrically connected to the reference electrode of the measurement module (e.g., the RL electrode in FIG. 7). In contact with the body of a user or a patient, a measurement electrode (e.g., one of the third wiring electrodes 831c) connected to the reference electrode (e.g., the fourth electrode 631d in FIG. 7) may be disposed at the same interval with respect to the remaining third wiring electrodes.

FIG. 14 is a view illustrating various shapes of an attachment pad 900 of a biological signal measurement device according to certain embodiments.

Referring to FIG. 14, the shapes of the attachment pad(s) 900 (e.g., the attachment pad 800 in FIG. 13), for example, there may be various extension directions or lengths of the extension portions (e.g., the extension portions 811b in FIG. 13). In an embodiment, an attachment pad(s) having a shape substantially based on a regular triangle may be attached to a portion in which there is a slight bending of the body. In another embodiment, in a portion to which the attachment pad(s) having a shape based on a regular triangle is difficult to stably attach (e.g., in a valley portion between the breasts or a portion below a rib), an attachment pad having a shape generally based on the letter “T” or “Y” may be easily attached. In the attachment pads having various shapes as described above, the structure of the coupling portion 911a may be substantially the same as the coupling member 701 in FIG. 9 or the coupling portion 811a in FIG. 13. For example, even if the shapes of the attachment pads 900 are different, the attachment pads may be coupled to a measurement device (e.g., the measurement module 400 in FIG. 5) so as to implement a biological signal measurement device (e.g., the biological signal measurement device 300 in FIG. 3).

According to certain embodiments, the attachment pad(s) may include at least one slit 911 so as to be stably attached to a bending portion of a body. For example, the slit(s) 911 may improve the flexibility of the attachment pad(s) 900. In some embodiments, the area of the attachment pad 900 (e.g., the pad body 801 in FIG. 13) may be reduced so as to improve the flexibility of the attachment pad. For example, as in the attachment pad indicated by reference numeral “901”, extension portions (e.g., the extension portion 811b in FIG. 13) may be formed to have a minimum area or a shape in which a third electrode or a fourth electrode (e.g., the second wiring electrode 831b or the third wiring electrode 831c in FIG. 12) may be disposed. In another embodiment, it is possible to improve the flexibility of the attachment pad by partially removing unnecessary portions of the pad body (e.g., the pad body 801 in FIG. 13). For example, as in the attachment pad indicated by reference numeral “902”, a flexible attachment pad having a regular triangle shape in appearance may be formed by partially removing the pad body in a region in which the wiring structure (e.g., the second wiring electrode 831b or the third wiring electrode 831c in FIG. 12) is not disposed.

As described above, according to certain embodiments, in the biological signal measurement device (e.g., the biological signal measurement device 300 in FIG. 3), it is possible to miniaturize a rigid module housing or measurement module (e.g., the measurement module 400 in FIG. 5) and to dispose measurement electrodes (the third wiring electrodes 831c in FIG. 12) on an attachment pad, which is flexible (or easily attachable to the user's body) (e.g., the attachment pad 800 in FIG. 12). For example, due to the flexibility of the attachment pad, it is easy to attach the attachment pad to a bent body part, and it is possible to secure a sufficient gap between the measurement electrodes. A module housing or a measurement module having a circuit device or the like therein may be electrically connected to the measurement electrodes via a wiring structure embedded in the attachment pad. According to an embodiment, the module housing or measurement module is capable of maintaining a stable coupling state with the attachment pad by magnetic force, which may facilitate replacement of the attachment pad. This magnetic force is generated by an electrode (e.g., the electrode 503 in FIG. 6) provided in the module housing or measurement module and an electrode (e.g., the first to fourth terminals 731a to 731d in FIG. 9) provided in the attachment pad. According to another embodiment, the number of measurement electrodes may be four. For example, a biological signal measurement device (e.g., the biological signal measurement device 300 in FIG. 3) may include one reference electrode and at least three measurement electrodes, in which two arbitrarily selected electrodes among three measurement electrodes may be paired (or may form a lead) so as to detect a biological signal. When the number of measurement electrodes is three, three electrode pairs, each including of two arbitrarily selected electrodes, may be formed. For example, one biological signal measurement device is capable of measuring a biological signal through three leads, and as the number of leads (e.g., electrode pairs) capable of measuring a biological signal increases, accuracy in measurement can be improved.

Hereinafter, a biological signal measurement method using an electronic device according to certain embodiments (e.g., the biological signal measurement device 300 in FIG. 3 or the measurement module or device 400 in FIG. 7) will be described with reference to FIG. 15. In describing the biological signal measurement method according to certain embodiments, it may be described that “a signal is sensed using the first to fourth electrodes 631a to 631d in FIG. 7”. However, as described above, it is noted that the measurement electrode to be brought into direct contact with the body of a user or a patient substantially means a third wiring electrode 831c of an attachment pad (e.g., the attachment pad 302 in FIG. 3 or the attachment pad 800 in FIG. 12). For example, the description, “a signal is sensed using the first to fourth electrodes 631a to 631d” means that “the first to fourth electrodes 631a to 631d are electrically connected the body of a user or a patient through the third wiring electrode 831c in FIG. 12 so as to sense a signal”.

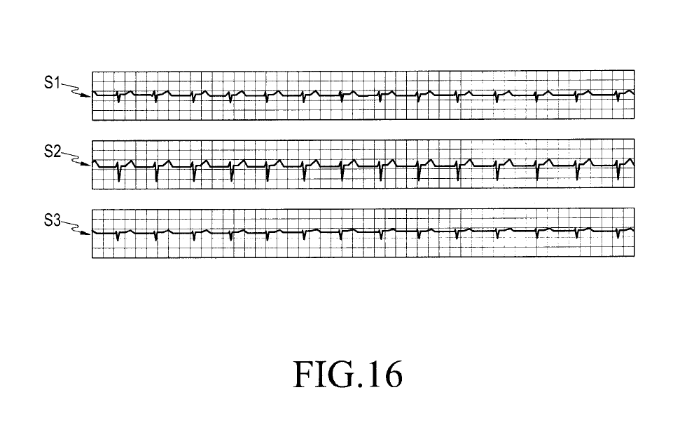

FIG. 15 is a flowchart for describing a biological signal measurement method (1500) using an electronic device according to certain embodiments. FIG. 16 is a view illustrating biological signals measured or determined through an electronic device according to certain embodiments.

Referring to FIG. 15, the method (1500) may include an operation of receiving (or identifying) an input or a request for a measurement (1501), sensing a signal on the basis of at least the received or identified request (1502), determining a biological signal through a combination of sensed signals (1503) and storing the biological signal (1504). These operations may be performed sequentially or in any order by the processor of an electronic device (e.g., the biological signal measurement device 300 in FIG. 3 or the measurement module 400 in FIG. 7).

According to certain embodiments, receiving an input or request for a measurement (1501) includes an operation in which a processor (e.g., the processor 120 in FIG. 1 or the control unit 211 in FIG. 2) receives or identifies a signal related to a measurement request (e.g., a request of measuring a biological signal such as an electrocardiogram), and may include receiving, by the processor, a signal generated by the operation of an operation unit (e.g., the operation unit 311a in FIG. 3). According to an embodiment, when an electronic device (e.g., the biological signal measurement device 300 in FIG. 3) is attached to the body of a user or a patient, and a signal is received through the electrodes (e.g., the first to fourth electrodes 631a-631d in FIG. 7), the processor may determine this initial signal to be an “input or request for measurement”.

According to certain embodiments, a signal (1502) may be sensed as generated from the body of a user or a patient on the basis of at least a request for measurement, using the first to fourth electrodes (e.g., the first to fourth electrodes 631a to 631d in FIG. 7). According to an embodiment, the processor may sense a first signal using the first electrode 631a and the fourth electrode 631d (e.g., a reference electrode) (1502a), may sense a second signal using the second electrode 631b and the fourth electrode 631d (1502b), and may sense a third signal using the third electrode 631c and the fourth electrode 631d (1502c).

According to certain embodiments, a biological signal may be determined through a combination of sensed signals (1503), including determining sensed first to third signals as a biological signal associated with the electrocardiogram. Further referring to FIG. 16, the processor (e.g., the processor 120 in FIG. 1) may determine (or set) the sensed first and second signals as a first biomedical signal S1 associated with the electrocardiogram. In an embodiment, the processor may determine (or set) the sensed second signal and third signal as a second biomedical signal S2 associated with the electrocardiogram, and may determine (or set) the sensed third signal and first signal as a third biological signal S3. For example, since a plurality of biological signals are capable of being detected through one electronic device, it is possible to improve the accuracy of biological signal measurement.

According to certain embodiments, the fourth electrode 631d is provided as a reference electrode for the first electrode 631a, the second electrode 631b, or the third electrode 631c, and the first biological signal may be determined by mutual comparison or by the combination of the signals sensed substantially through the first electrode 631a and the second electrode 631b (with reference to the potential of the fourth electrode 631d). For example, the first biological signal may be determined on the basis of the signal(s) sensed through the electrode pair or the lead including of the first electrode 631a and the second electrode 631b. In some embodiments, the second biological signal may be determined on the basis of the signal(s) sensed through the electrode pair or the lead including of the second electrode 631b and the third electrode 631c. In another embodiment, the third biological signal may be determined on the basis of the signal(s) sensed through the electrode pair or the lead including of the third electrode 631c and the first electrode 631a.

According to certain embodiments, the operation of storing a biological signal (1504) is the operation of storing a biological signal in a memory (e.g., the memory 130 in FIG. 1), and the processor may be configured to store at least one of determined signals in the memory as at least a piece of measurement information on the electrocardiogram. In some embodiments, at least one of the first biological signal, the second biological signal, and the third biological signal may be a piece of measurement information associated with the electrocardiogram, and the processor may store the biological signals as described above in the memory as a piece of the measurement information associated with the electrocardiogram. According to an embodiment, the memory is mounted on the electronic device (e.g., the biological signal measurement device 300 in FIG. 3 or the measurement module 400 in FIG. 7) and may be functionally connected to the processor. In another embodiment, the memory may be mounted on another electronic device or server (e.g., the electronic device 102 or 104 or the server 108 in FIG. 1) that is connected to the electronic device through a direct (e.g., wired) communication channel or a wireless communication channel. For example, in the operation of storing a biological signal (1504), the processor may be configured to transmit at least a portion of the measurement information directly to another electronic device or server connected via a communication channel or wireless communication channel.

According to certain embodiments, a biological signal measurement device or an electronic device (e.g., the biological signal measurement device 300 in FIG. 3) may include: a housing (e.g., a module housing 301 in FIG. 3 or a measurement module 400 in FIG. 5); a first electrode (e.g., the first electrode 631a in FIG. 7) disposed on one face of the housing; a second electrode (e.g., the second electrode 631b in FIG. 7) disposed on the one face of the housing; a third electrode (e.g., the third electrode 631c in FIG. 7) disposed on the one face of the housing; a fourth electrode (e.g., the fourth electrode 631d in FIG. 7) disposed on the one face of the housing; and a processor (e.g., the processor 120 in FIG. 1), such that the processor may be configured to: identify a request associated with measurement of an electrocardiogram in a living body; sense a first signal using the first electrode and the fourth electrode based at least on the request; sense a second signal using the second electrode and the fourth electrode based at least on the request; sense a third signal using the third electrode and the fourth electrode based at least on the request; determine the first signal and the second signal as a first biological signal associated with the electrocardiogram, the second signal and the third signal as a second biological signal associated with the electrocardiogram, the third signal and the first signal as a third biological signal associated with the electrocardiogram; and store at least one of the first biological signal, the second biological signal, and the third biological signal in a memory (e.g., the memory 130 in FIG. 1) functionally connected thereto as at least a part of measurement information on the electrocardiogram.

According to certain embodiments, the electronic device may further include an attachment pad (the attachment pad 302 in FIG. 3) detachably provided on one face of the housing, and the attachment pad may include a first terminal (e.g., the first terminal 731a in FIG. 9) configured to be in electrical contact with or to be electrically connected to the first electrode, a second terminal (e.g., the second terminal 731b in FIG. 9) configured to be in electrical contact with or to be electrically connected to the second electrode, a third terminal (e.g., the third terminal 731c in FIG. 9) configured to be in electrical contact with or to be electrically connected to the third electrode, and a fourth terminal (e.g., the fourth terminal 731d in FIG. 9) configured to be in electrical contact with or to be electrically connected to the fourth electrode.

According to certain embodiments, the attachment pad may include an elastic material.

According to certain embodiments, the attachment pad may further include a coupling member (e.g., the coupling member 323 in FIG. 3) coupled to one face of the housing by magnetic force, and the coupling member may be coupled so as to enclose at least a portion of the housing by the magnetic force.

According to certain embodiments, the attachment pad may further include a coupling member coupled to one face of the housing by magnetic force, and the first terminal, the second terminal, the third terminal, or the fourth terminal may be arranged in the coupling member.

According to certain embodiments, the attachment pad may further include: first wiring electrodes (e.g., the first wiring electrodes 831a in FIG. 12) electrically connected to the respective ones of the first terminal, the second terminal, the third terminal, and the fourth terminal; second electrodes (e.g., the second electrodes 831b in FIG. 12) extending from the respective ones of the first wiring electrodes; and third electrodes (e.g., the third wiring electrodes 831c in FIG. 12) provided in the respective ones of the ends of the second wiring electrodes.

On the attachment pad, the third wiring electrodes may be exposed in a direction different from a direction of the first terminal, the second terminal, the third terminal, or the fourth terminal.

According to certain embodiments, an arrangement interval of the third electrodes may be larger than an arrangement interval of the first terminal, the second terminal, the third terminal, and the fourth terminal.

According to certain embodiments, the third wiring electrodes may be formed of a conductive hydrogel.

According to certain embodiments, the attachment pad may further include an adhesive layer (the second adhesive layer 801c in FIG. 12) having an exposed face on which the third wiring electrodes are disposed.

According to certain embodiments, the attachment pad may further include a coupling member coupled to the housing by magnetic force, a coupling portion in which the coupling member (e.g., the coupling portion 811a in FIG. 13) is disposed, and extension portions (the extension portions 811b in FIG. 13), each extending from the coupling portion, and the third wiring electrodes may be disposed on the respective ones of the extension portions.

According to certain embodiments, a first face of the housing may have a circular shape, and the first electrodes may be arranged at equal angular intervals in a circumferential direction on the first face of the housing.

According to certain embodiments, the electronic device may further include at least one first alignment key (e.g., the first alignment key 633 in FIG. 7) protruding or recessed in the first face of the housing.

According to certain embodiments, at least one of the first electrode, the second electrode, the third electrode, and the fourth electrode may include: a first electrode plate (e.g., the first electrode plate 533 in FIG. 6) including an accommodation recess (e.g., the accommodation recess 533a in FIG. 6) disposed in an inner face and a flange (the flange 533b in FIG. 6) provided around the accommodation recess and supported by or fixed to an inner face of the housing, the flange being made of an electrically conductive material; and a permanent magnet (e.g., the permanent magnet 535 in FIG. 6) accommodated in the accommodation recess.

In the outer face of the first electrode plate, a region corresponding to the accommodation recess is exposed to a first face of the housing (e.g., the housing 601 in FIG. 7).

According to certain embodiments, the flange may be attached or fixed to the inner face of the housing.

According to certain embodiments, the permanent magnet may be fixed in the accommodation recess by magnetic force.

According to certain embodiments of the present disclosure, an attachment pad (e.g., the attachment pad 800 in FIG. 12) for an electronic device such as a biological signal measurement device may include: a pad body (e.g., the pad body 801 in FIG. 12) including a coupling portion (e.g., the coupling portion 811a in FIG. 13) to which a module housing or a measurement module is coupled, and extension portions (e.g., the extension portions 811b), each extending in a direction away from the coupling portion; a coupling member (e.g., the coupling member 701 in FIG. 9) provided in the coupling portion on a first face of the pad body; a plurality of terminals provided in the coupling member; and a plurality of measurement electrodes, which are disposed on the respective ones of the extension portions on a second face of the pad body, which is directed away from the first face of the pad body.

Each of the measurement electrodes may be electrically connected to one of the terminals.

According to certain embodiments, the coupling member may include at least one alignment key protruding from or recessed into a face on which the terminals are arranged.

According to certain embodiments, the pad body may further include at least one slit disposed at an edge of at least one of the extension portions.

According to certain embodiments, the terminals may generate an attractive force of magnetic force when the terminals are positioned adjacent to the permanent magnet.

According to certain embodiments, the measurement electrodes may be formed of a conductive hydrogel, and the interval between the measurement electrodes may be larger than the interval between the terminals.

In the foregoing detailed description, specific embodiments of the present disclosure have been described. However, it will be evident to a person ordinarily skilled in the art that various modifications may be made without departing from the present disclosure. For example, although a specific embodiment discloses a biological signal measurement device, the attachment pad for a biological signal measurement device may also be utilized for a treatment purpose such as low-frequency treatment.

What is claimed is:

1. An electronic device comprising:
a housing;
a plurality of electrodes disposed on one face of the housing; and
at least one processor; and
memory storing programming instructions executable by the at least one processor to cause the electronic device to:
in response to receiving a request for an electrocardiogram measurement:
detect a first signal using a first electrode and a fourth electrode from among the plurality of electrodes,
detect a second signal using a second electrode and the fourth electrode,
detect a third signal using a third electrode and the fourth electrode, and
storing in the memory the first signal and the second signal as a first biological signal, the second signal and the third signal as a second biological signal and the third signal and the first signal as a third biological signal in association with the requested electrocardiogram measurement.

2. The electronic device of claim 1, wherein the electronic device further comprises an attachment pad detachably provided on the one face of the housing,
wherein the attachment pad includes:
a first terminal electrically coupled with the first electrode;
a second terminal electrically coupled with the second electrode;
a third terminal electrically coupled with the third electrode; and
a fourth terminal electrically coupled with the fourth electrode.

3. The electronic device of claim 2, wherein the attachment pad includes an elastic material.

4. The electronic device of claim 2, wherein the attachment pad further includes a coupling member coupled to the one face of the housing by magnetic force, and
wherein the coupling member encloses at least a portion of the housing by the magnetic force.

5. The electronic device of claim 4, wherein
the first terminal, the second terminal, the third terminal, and the fourth terminal are disposed within an interior space of the coupling member.

6. The electronic device of claim 2, wherein the attachment pad further includes:
first wiring electrodes electrically coupled to each of the first terminal, the second terminal, the third terminal, and the fourth terminal, respectively;
second wiring electrodes extending from each of the first wiring electrodes; and
third wiring electrodes, each provided on an end of each of the second wiring electrodes, and
wherein the third wiring electrodes are exposed through openings defined in the attachment pad in a first direction oriented away from at least one of the first terminal, the second terminal, the third terminal, and the fourth terminal.

7. The electronic device of claim 6, wherein the third electrodes are disposed in a first arrangement, and the first, second, third and fourth terminals are disposed in a second arrangement,
wherein the first arrange includes a first interval distance substantially uniform between each electrode of the third electrodes, and the second arrangement includes a second interval distance substantially uniform between each of the first, second, third and fourth terminals, and
wherein the first interval distance is greater than the second interval distance.

8. The electronic device of claim 6, wherein the third wiring electrodes comprise a conductive hydrogel.

9. The electronic device of claim 6, wherein the attachment pad further includes an adhesive layer, the adhesive layer including an exposed face through which the third wiring electrodes are disposed.

10. The electronic device of claim 6, wherein the attachment pad includes:
a coupling member coupled to the housing by magnetic force;
a coupling portion in which the coupling member is disposed; and
extension portions each extending from the coupling portion, and
wherein the third wiring electrodes are disposed on each of the extension portions respectively.

11. The electronic device of claim 1, wherein the one face of the housing includes a circular shape, and the first electrodes are circumferentially arranged at equal angular intervals around the circular shape of the one face of the housing.

12. The electronic device of claim 1, further comprising:
at least one first alignment key protruding or recessed in the one face of the housing.

13. The electronic device of claim 1, wherein at least one of the first electrode, the second electrode, the third electrode, and the fourth electrode includes:
a first electrode plate including an accommodation recess disposed in an inner face,
a flange disposed around the accommodation recess and coupled to an inner face of the housing, the flange including an electrically conductive material and; and
a permanent magnet disposed within the accommodation recess,
wherein a region of an outer face of the first electrode plate and corresponding to the accommodation recess is exposed to a first face of the housing.

14. The electronic device of claim 13, wherein the flange is coupled to the inner face of the housing.

15. The electronic device of claim 13, wherein the permanent magnet is fixed in the accommodation recess by magnetic force.

16. An attachment pad in a biological signal measurement device, the attachment pad comprising:
a pad body including a coupling portion for coupling to a module housing or a measurement module, and extension portions each extending away from the coupling portion;
a coupling member provided in the coupling portion and disposed on a first face of the pad body;
a plurality of terminals disposed in the coupling member; and
a plurality of measurement electrodes, each measurement electrode disposed on each of the extension portions respectively, each of the extension portions disposed on a second face of the pad body oriented away from the first face of the pad body,
wherein each of the measurement electrodes is electrically connected to one of the plurality of terminals.

17. The attachment pad of claim 16, wherein the coupling member includes at least one alignment key protruding from or recessed into a face on which the terminals are disposed.

18. The attachment pad of claim 16, wherein the pad body further defines at least one slit, the at least one slit disposed at an edge of at least one of the extension portions.

19. The attachment pad of claim 16, wherein the terminals generate an attractive magnetic force when the terminals are positioned adjacent to a permanent magnet.

20. The attachment pad of claim 16, wherein the measurement electrodes comprise a conductive hydrogel, and an interval between the measurement electrodes is larger than an interval between the terminals.
